(19) United States
(12) Patent Application Publication
(10) Pub. No.: US 2005/0049311 A1
(43) Pub. Date: Mar. 3, 2005

(54) Medicinal products comprising prostaglandin compositions and methods of packaging such compositions

(75) Inventors: David Stephen Baker, Encinitas, CA (US); Paramita Bandyopadhyay, Portage, MI (US); Satish Kumar Singh, Wildwood, MO (US)

Correspondence Address: AGOURON PHARMACEUTICALS, INC., 10350 NORTH TORREY PINES ROAD, LA JOLLA, CA, 92037, US

(73) Assignee: Pharmacia & Upjohn Company

(21) Appl. No.: 10/932,405

(22) Filed: Sep. 2, 2004

Related U.S. Application Data
(60) Provisional application No. 60/499,853, filed on Sep. 3, 2003.

(51) Int. Cl. A61K 31/505; A61K 31/557

(52) U.S. Cl. 514/573; 514/256; 514/374; 514/438; 206/528

(57) ABSTRACT

A packaged medicinal product comprising (a) a pharmaceutical composition, and b) a plastic container. The composition comprises a prostaglandin, or an analogue or a derivative thereof. The plastic container has a surfactant barrier layer on an inner surface of the container wall and is filled with the pharmaceutical composition. There is also provided method of packaging such a pharmaceutical composition. The method comprises the steps of (a) providing such a plastic container, and (b) filling the container with the pharmaceutical composition.

This application claims the benefit of U.S. Provisional Patent Application No. 60/499,853 filed on Sep. 3, 2003, hereby incorporated in its entirety for all purposes.

### FIELD OF THE INVENTION

The present invention relates generally to packaging of medicinal products. In particular, the present invention relates to storage-stable containers suitable for pharmaceutical compositions, especially pharmaceutical prostaglandin compositions.

### BACKGROUND OF THE INVENTION

Pharmaceutical compositions comprising one or more active ingredients must possess certain levels of stability and purity in order to be suitable for safe and efficacious administration to patients. The pharmaceutical composition may also contain other components, e.g. excipients, flavoring agents and preservatives. Such compositions are considered stable if the concentration of active ingredient(s) can be maintained at the level specified on the label for the maximum anticipated shelf life under given environmental conditions. A pharmaceutical composition is considered unstable when the potency of the active ingredient(s) is lost.

The potency of a medicinal product may decline over time during storage due to various reasons, such as degradation of the active ingredient(s), reaction of the active ingredient(s) with excipients or container materials, leaching of the active ingredient(s) through the container wall, and absorption/adsorption of the active ingredient(s) into/onto the container wall. Similarly, the purity of a pharmaceutical composition may also change during storage due to leaching of chemicals into the composition from the container materials, from the labels on the containers, or from the environment where the packaged medicinal product is stored. Thus, the containers used for packaging pharmaceutical compositions can significantly affect the stability and purity of the compositions contained therein. Preservatives, for example, may also permeate the container wall, and as a result, their preservative value is diminished. The solvent of the composition may also permeate the container wall, which will increase the concentration of the active ingredient and of the other components of the composition.

One example of a group of active ingredients is prostaglandins, and analogues and derivatives thereof. Prostaglandins such as, but not limited to, latanoprost, are susceptible to adsorption/absorption by plastic packaging components, and this adsorption/absorption decreases the potency of the compositions upon storage. Prostaglandins are relatively unstable medical agents and are used for treatment of various conditions, including glaucoma, which is an eye disorder afflicting various mammals, including primates and humans, and is characterized in that it increases intraocular pressure (ocular hypertension).

Containers commonly used for medicinal products comprising prostaglandins include glass containers, polypropylene containers, and polyethylene containers (see e.g. U.S. Pat. No. 6,235,781). However, since glass containers are rigid and not squeezable, they are not very suitable for medicinal preparations which are conveniently dispensed on a drop-by-drop basis. Typical user-friendly containers, dispensers, or bottles for medicinal preparations are formed from e.g. polyethylene, polypropylene, or polyethylene terephthalates (PET), which in most instances provide a suitable combination with a pharmaceutical preparation, resulting in a packaged medicinal product that is user-friendly for dispensing of the pharmaceutical preparation on a drop-by-drop basis.

The ophthalmic products XALATAN® (comprising approximately 50 μg/ml latanoprost) and XALCOM® (comprising approximately 50 μg/ml latanoprost and 5 mg/ml timolol) are packaged in bottles. The bottle is typically made of clear low-density polyethylene with a clear linear low-density polyethylene dropper tip, a high-density polyethylene screw cap, and a clear low-density polyethylene overcap. Unopened bottles are stored at 2-8° C. Upon storage at the recommended temperature, the concentrations of latanoprost and latanoprost/timolol, respectively, are stably maintained in the products.

Plastic containers have some drawbacks since the active agent, depending on the choice of plastic material employed for the container, can be absorbed and/or adsorbed by the container material. This absorption/adsorption decreases the potency of the formulations during storage. In order to reduce or prevent the absorption and/or adsorption of certain prostaglandins, the plastic containers comprising the pharmaceutical compositions are commonly kept in refrigerators until opened, and once opened, they can be kept in room temperature and used within a limited period of time, typically 1-2 months. The requirement that the ophthalmic preparation is to be kept refrigerated reduces the availability of the treatment in countries where such storage conditions are less available or reliable. Furthermore, even where available, refrigeration of the preparation increases the cost of the treatment for the patient, and thus, further reduces its availability to those in need.

Moreover, many prostaglandins have a limited solubility in water. In order to improve the solubility of the prostaglandins, such compositions may also contain surfactants, as is the case with unoprostone isopropyl ester in RESCULA®, commercially available from Novartis.

WO 00/03736 discloses aqueous compositions containing one or more prostaglandins and at least one surfactant in order to help solubilize or disperse the prostaglandin in the composition. The surfactants that are present in the composition also inhibit or prevent the adsorption of the prostaglandin on to the container walls. Typically, the container walls are made of polypropylene.

WO 4,501,793 discloses polyolefinic microporous materials prepared by impregnating a normally hydrophobic polyolefinic microporous substrate material with a solution of a non-ionic alkylphenoxy poly(ethyleneoxy) ethanol surfactant. The hydrophilic polyolefinic microporous materials are said to be useful as battery separators and as membranes for use in various processes.

Despite these advances, there is still an unmet need for a method for storing such pharmaceutical compositions over periods of time without refrigeration. There is also a need for a packaged medicinal product and method for packaging pharmaceutical compositions, which can increase the stability of the pharmaceutical composition or prevent the loss of an ingredient of the composition.

Furthermore, there is an urgent need for containers for pharmaceutical prostaglandin compositions, and methods of packaging such compositions, which can increase the storage stability of the composition by preventing the loss of the active ingredient of the preparation.

### SUMMARY OF THE INVENTION

It is an object of the present invention to provide a packaged medicinal product that is suitable for storage of a pharmaceutical composition comprising a prostaglandin over periods of time without refrigeration.

It is a further object of the invention to provide a packaged medicinal product that is suitable for storage of a pharmaceutical composition comprising a prostaglandin and which can provide a desired stability of the prostaglandins.

It is also an object of the invention to provide a packaged medicinal product that is suitable for storage of a pharmaceutical composition comprising a prostaglandin and can prevent or diminish the loss of the prostaglandin.

It is an object of the invention to provide an improved method of packaging a pharmaceutical composition comprising a prostaglandin.

It is another object of the invention to provide a method of stabilizing prostaglandin, contained in a pharmaceutical composition.

It is yet another object of the invention to provide a method of preventing sorptive loss of an ingredient in a pharmaceutical composition comprising a prostaglandin to the wall of a plastic container containing the pharmaceutical composition.

Pharmaceutical compositions that are suitable for use in the invention are not limited by their usage or indication. It is advantageous, however, that the composition is useful for treatment of ophthalmic conditions, such as glaucoma.

An embodiment of the invention provides a packaged medicinal product comprising:
a) a pharmaceutical composition comprising a prostaglandin, or an analogue or a derivative thereof; and
b) a plastic container having a surfactant barrier layer on an inner surface of the container wall, wherein the container is filled with the pharmaceutical composition.

In another embodiment, the plastic containers are coated with a layer of surfactants, or if plastic containers are impregnated with surfactants, their absorption and/or adsorption of prostaglandin is prevented and thus, storage-stable medicinal products comprising prostaglandin compositions are obtained.

In yet another embodiment, there is provided a method of packaging a pharmaceutical composition comprising a prostaglandin, or an analogue or a derivative thereof, said method comprising the steps of providing a plastic container having a surfactant barrier layer on an inner surface of the container wall; and filling the container with the pharmaceutical composition.

According to yet another embodiment, there is provided a method of stabilizing a prostaglandin, or an analogue or a derivative thereof, contained in a pharmaceutical composition, said method comprising packaging the pharmaceutical composition in a plastic container having a surfactant barrier layer on an inner surface of the container wall.

According to yet another embodiment, there is provided a method of preventing sorptive loss of a prostaglandin, or an analogue or a derivative thereof, from a pharmaceutical composition to the wall of a plastic container containing the pharmaceutical composition, said method comprising packaging the pharmaceutical composition in the plastic container, said container having a surfactant barrier layer on an inner surface of the container wall.

In certain embodiments of the medicinal product and the methods according to the invention, said pharmaceutical composition is an aqueous liquid.

In yet another embodiment of the medicinal product and methods, the concentration of said prostaglandin, or analogue or derivative thereof in said pharmaceutical composition is in the range of 0.001%-0.5% (w/v), preferably in the range of 0.005%-0.1% (w/v).

In certain embodiments of the medicinal product and methods, said prostaglandin, or analogue or derivative thereof, is selected from the group comprising physiologically acceptable derivatives of prostaglandin F (PGF), such as those described therein. Specific prostaglandins, or analogues or derivatives thereof, that are useful in the medicinal product and the methods according to the invention are preferably selected from the group comprising 17-phenyl-18,19,20-trinor derivatives, 13,14-dihydro-17-phenyl-18,19,20-trinor derivatives, 16-phenoxy-17,18,19,20-tetranor derivatives, or 16-phenyl-17,18,19,20-tetranor derivatives.

In particular embodiments of the medicinal product and methods, said plastic container is made of a polymeric material selected from the group comprising low-density polyethylene (LDPE), high-density polyethylene (HDPE), polypropylene (PP) or blends containing two or more of the following: LDPE, HDPE, PP, plastomers, and polycarbonates.

In specific embodiments of the medicinal product and methods, said polymeric material is a blend of
(a) 0.1-8 wt % of LDPE or HDPE, or a combination of LDPE and HDPE, and
(b) 92-99.9 wt % of PP.

In certain embodiments of the medicinal product and methods, said plastic container is a small volume bottle.

In particular embodiments of the medicinal product and methods, said surfactant barrier layer comprises a surfactant selected from the group comprising POE-80 sorbitan laurate, the Brij series, e.g. polyoxyethylene 20 stearyl ethers (also known as the Brij series of surfactants dominated by the weight of PEG used to form the ether linkage) (Brij 78), Brij 30, Brij 35, Brij 52, Brij 56, Brij 58, Brij 72, Brij 721, Brij 76, Brij 92, Brij 96, and Brij 98, the Tween®/Polysorbate series (poloxyethylene sorbitan esters), e.g. Tween® 20 (polyoxyethylene [20] sorbitan monolaurate), Tween® 40 (polyoxyethylene sorbitan monopalmitate), Tween® 60 (polyoxyethylene sorbitan monostearate), and Tween® 80 (polyoxyethylene sorbitan monooleate), phospholipids and their derivatives, ethoxylated alkyl phenol, pegylated hydrogenated castor oils (e.g. CREMOPHOR® EL (PEG-35 castor oil), CREMOPHOR® RH (PEG-40 Hydrogenated Castor Oil) and CREMOPHOR® CO-40 (PEG-40 Hydrogenated Castor Oil) each commercially available from BASF Chemical Company), stearoyl macrogol esters, glyceryl stearates, Estasan series (medium chain tri-glycerides), sorbitan fatty acid esters.

In specific embodiments of the medicinal product and methods, said surfactant is selected from the group comprising Polysorbates (e.g. polysorbate 20 (polyoxyethylene [20] sorbitan monolaurate) or 80 (polyethylene sorbitan monooleate)), Brij series (e.g. Brij 35, 52 or 78) and hydrogenated pegylated castor oil derivatives, e.g. CREMOPHOR® EL, CREMOPHOR® RH and CREMOPHOR® CO-40.

In certain embodiments of the medicinal product and methods, said pharmaceutical composition further comprises a surfactant. In another embodiment, said surfactants in the pharmaceutical composition and on said inner surface of the container wall, respectively, have the same type of surface charge. In particular embodiments, a single type of surfactant is used both in the pharmaceutical composition and on said inner surface of the container wall, respectively.

In particular embodiments of the medicinal product and methods, said pharmaceutical composition is for ophthalmic use.

According to another embodiment, there is provided a novel use of a surfactant for preventing sorptive loss of an ingredient in a pharmaceutical composition comprising a prostaglandin, or an analogue or a derivative thereof, to a wall of a plastic container containing the pharmaceutical composition, by providing a surfactant barrier layer comprising said surfactant on an inner surface of the container wall.

According to another aspect of the invention, there is provided a method of packaging a pharmaceutical composition comprising a prostaglandin, or an analogue or a derivative thereof. The method comprises the steps of:
a) providing a plastic container having a surfactant barrier layer on an inner surface of the container wall; and
b) filling the container with the pharmaceutical composition.

As used therein, the term “prostaglandin” or “PG” shall refer to prostaglandins and derivatives and analogues thereof, including pharmaceutically acceptable salts and esters, except as otherwise indicated by context. Prostaglandins may be classified according to their 5-membered ring structure, using a letter designation. Prostaglandins may be further classified based on the number of unsaturated bonds in the side chain. The prostaglandins that may be utilized in the present invention include all pharmaceutically acceptable prostaglandins, their derivatives and analogues, and their pharmaceutically acceptable esters and salts. Such prostaglandins include the natural compounds POE₁, PGE₂, PGE₃, PGF₁ₐ, PGF₂ₐ, PGF₃ₐ, PGD₂, and PGI₂ (prostacyclin), as well as analogues and derivatives of these compounds which have similar biological activities.

Analogues of the natural prostaglandins include, but are not limited to, compounds resulting from modifications of the omega chain (e.g. 18,19,20-trinor-17-phenyl, 17,18,19,20-tetranor-16-phenoxy), which enhance selectivity of action and reduce ocular side-effects. The “nor” derivative denotes (a) elimination of one methylene group from a chain, the highest permissible locant being used, or (b) contraction of a ring by one CH₂ unit, the locant being the capital letter identifying the ring. Elimination of two or more methylene groups may be denoted by the prefixes “dinor-”, “trinor-”, “tetranor-” and so on.
Such prostaglandin derivatives are physiologically acceptable derivatives of prostaglandin F (PGF), in which the omega chain has the formula (I):

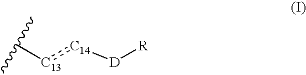

Most preferably, the prostaglandin derivatives may be 17-phenyl-18,19,20-trinor derivatives, 13,14-dihydro-17-phenyl-18,19,20-trinor derivatives, 16-phenoxy-17,18,19,20-tetranor derivatives, or 16-phenyl-17,18,19,20-tetranor derivatives.

Derivatives of these prostaglandins include all pharmaceutically acceptable salts, esters (alkyl esters, such as isopropyl esters) and amides (lower alkyl amides, such as N-diethyl amide) that may be attached to the 1-carboxyl group or any of the hydroxyl groups of the prostaglandin by use of the corresponding alcohol or organic acid reagent, as appropriate. It should be understood that the terms “analogues” and “derivatives” include compounds that exhibit functional and physical responses similar to those of prostaglandins per se.

Specific examples of prostaglandins that are useful in the present invention include the following compounds:
1. 17-phenyl-18,19,20-trinor-PGF₂ₐ-isopropyl ester;
2. 15-(R)-17-phenyl-18,19,20-trinor-PGF₂ₐ-isopropyl ester;
3. 16-phenyl-17,18,19,20-tetranor-PGF₂ₐ-isopropyl ester;
4. 20-pentanor-13-prostynoic acid;
5. latanoprost;
6. cloprostenol isopropyl ester;
7. (5Z)-(9S, 11R, 15S)-15-cyclohexyl-9,11,15-trihydroxy-16,17,18,19,20-pentanor-5-prostenoic acid isopropyl ester;
8. (5Z, 13E)-(9S,11R,15R)-9,11,15-trihydroxy-16-(3-chlorophenoxy)-17,18,19,20-tetranor-5,13-prostadienoic acid amide;
9. PGF₂ₐ isopropyl ester;
10. fluprostenol isopropyl ester;
11. isopropyl [2R(1E,3R),3S(4Z),4R]-7-[tetrahydro-2-[4-(3-chlorophenoxy)-3-hydroxy-1-butenyl]-4-hydroxy-3-furanyl]-4-heptenoate;
12. 15-keto latanoprost;
13. bimatoprost;
14. unoprostone isopropyl;
15. 15-deoxy-15-fluoro-16-phenoxy-17,18,19,20-tetranor-PGF₂ₐ isopropyl ester;
16. 15-deoxy-15-fluoro-16-(3-chlorophenoxy)-17,18,19,20-tetranor-PGF₂ₐ isopropyl ester;
17. 15-deoxy-15-fluoro-16-(3-trifluoromethylphenoxy)-17,18,19,20-tetranor-PGF₂ₐ isopropyl ester;
18. 15-deoxy-15-difluoro-16-phenoxy-17,18,19,20-tetranor-PGF₂ₐ isopropyl ester;
19. 15-deoxy-15-difluoro-16-(3-chlorophenoxy)-17,18,19,20-tetranor-PGF₂ₐ isopropyl ester;
20. 15-deoxy-15-difluoro-16-(3-trifluoromethylphenoxy)-17,18,19,20-tetranor-PGF₂ₐ isopropyl ester;
21. 15-deoxy-15-difluoro-16-phenoxy-17,18,19,20-tetranor-PGF₂ₐ isopropyl ester; and
22. DE-085.

The terms “sorption” and “sorptive”, as used herein, refers to absorption or adsorption or a combination of absorption and adsorption.

The term “medium chain tri-glycerides” as used herein, refers to a tri-glyceride with a carbon chain from C₅-C₁₆.

The terms “stabilizing” and “stability provided by a material”, as used herein, refer to what degree of sorption, including absorption and/or adsorption, the material exhibits with respect to a substance exposed to the material. In particular, the term relates to the degree of sorption of an active ingredient, such as a prostaglandin, of a pharmaceutical composition exhibited by a material.

The term “package material”, as used herein, means any material that is suitable for containers for pharmaceutical compositions. Such pharmaceutically acceptable materials include, but are not limited to materials comprising one or more polyolefins. Examples of suitable package materials for containers for pharmaceutical compositions include materials comprising ethylene or propylene monomers, or both.

The term “active ingredient(s)”, as used herein, comprises the therapeutically active compound(s) that provides a desired effect upon administration of a therapeutically effective amount thereof to a human or an animal in need thereof. The active ingredient is commonly provided in a suitable pharmaceutical composition.

The terms “therapeutically effective concentration” and “therapeutically effective amount”, as used herein, means an amount, or a concentration that upon administration of the proper volume results in such an amount, which upon administration thereof to a human or an animal in need thereof exerts desirable therapeutic effects.

In accordance with convention used in the art, the symbol [symbol] is used in structural formulas herein to depict the bond that is the point of attachment of the moiety or substituent to the core or backbone structure of the prostaglandin molecule.

### DETAILED DESCRIPTION OF THE INVENTION

The inventive idea resides in an improved packaged medicinal product. The term “medicinal product”, as used therein, refers to packaged products, which upon use provides biological, physiological, pharmacological, or chemical activities that are beneficial for animals or humans in normal or pathological conditions, such as diagnosis, prognosis, treatment, prophylaxis, therapy, or for animal production.

Typically, the medicinal product encompasses a pharmaceutical composition comprising one or more active ingredients, packaged in a suitable container. The term “pharmaceutical composition”, as used therein, refers to a composition containing one or more active ingredients in a suitable concentration for providing the desired biological, physiological, pharmacological, or chemical activities that are beneficial for animals or humans in normal or pathological conditions, such as diagnosis, prognosis, treatment, prophylaxis, therapy, or for animal production. The pharmaceutical composition may also contain other components, e.g. excipients, flavoring agents and preservatives.

Any suitable pharmaceutical composition may be may be incorporated into the present invention. Additionally, any suitable pharmaceutical composition comprising a prostaglandin, or an analogue or a derivative thereof, may be incorporated into the present invention. Thus, for the present invention, the pharmaceutical composition is not limited by its specific application, physical form, formulation, or specific dosage form. For example, the composition can be in the form of a powder, capsule, tablet, or liquid and any other form, specifically, liquid compositions are suitable for use in the invention. The liquid composition may be a suspension, solution, emulsion, microemulsion, gel, or in other liquid form, and can be aqueous or non-aqueous.

Without being bound to any particular theory, it is envisaged that the medicinal product according to the invention exhibits a more hydrophilic inner surface of the container than in the corresponding known containers that lack the inventive surfactant barrier layer. It is believed that the tendency of the hydrophobic prostaglandin to adhere to the inner wall of the container lessens upon increased hydrophilicity of the inner wall. In known prostaglandin preparations, surfactant may be included in the composition in order to increase the solubility of the hydrophobic prostaglandin by making the surface of the prostaglandin/surfactant complex more hydrophilic. Thus, the purpose of the use of surfactants in known medicinal products is related to making the prostaglandin/surfactant complex more hydrophilic. In contrast to the known medicinal products, the purpose of the use of surfactant in the present invention is to make the inner surfaces of the containers more hydrophilic.

In addition, if the pharmaceutical prostaglandin composition comprises a surfactant, the composition may be rendered more stable in that the likelihood of loss of surfactant itself to the container surface according to the invention is decreased, thereby preventing the solubilized material from falling out of solution. Furthermore, if a surfactant used in a composition is charged, a container surface according to the invention that is pre-saturated with the same surfactant, or a surfactant having the same charge, will repel the charged micelles that are solubilizing the drug and prevent both drug and surfactant from partioning into the container wall.

All of the foregoing compounds are known. The initial concentration of the selected active ingredient in said composition may vary depending on the therapeutically effective concentration. For example, the concentration of the selected active ingredient may be in the range of from 0.001% to 0.5% (w/v), such as from 0.005% to 0.1% (w/v). In certain preferred embodiments, the active ingredient is latanoprost. Therapeutically effective concentrations of latanoprost may be in the range of 1-1000 μg/ml, preferably 10-100 μg/ml.

The plastic container according to the invention is made of any pharmaceutically acceptable plastic material. Thus, the container may comprise any suitable polyolefin known to those skilled in the art. Illustrative examples of suitable polyolefins include polyethylene (PE), polypropylene (PP), poly-3-methyl butene-l, poly-4-methyl pentene-1, copolymers of ethylene, propylene, 3-methyl butene-l, or 4-methyl pentene-l with each other or with minor amounts of other olefins, e.g. copolymers of ethylene and propylene.

Polyethylene is commonly divided into classes based on its density. Classes commonly used include high-density polyethylene (HDPE), medium-density polyethylene (MDPE), and low-density polyethylene (LDPE). This list of classifications should not be considered as a standard or a complete list of classifications. Given these rather loose classifications, polymer characteristics vary among multiple producers of a given class of polyethylene, or among multiple grades of a given class by one producer.

The container according to the invention can be made of polyethylene of any density, a blend of polyethylenes of various densities, or a blend of polyethylene with other materials. For medicinal preparations that are desirably packaged in squeezable containers, particularly ophthalmic medications, it is advantageous that the container of the invention is made of a material comprising LDPE. Depending on the desired level of squeezability of the container, the relative content of LDPE in the container materials can be adjusted accordingly. Generally, containers made of LDPE are more readily squeezable than containers made of MDPE or HDPE. Similarly, containers made of materials having a relatively high content of LDPE are more readily squeezable than containers made of materials having a relatively low content of LDPE.

Alternatively, the container according to the invention can be made of polypropylene or a blend of polypropylene with other materials.

The polyolefinic material may also include small amounts (e.g. less than about 20% by weight) of other materials which may be copolymerized or blended therewith, but which do not substantially adversely affect the characteristics of the polyolefinic material. Preferred polymeric materials for the containers are HDPE, MDPE, LDPE, and PP, or blends thereof. Other preferred blends include, but are not limited to, HDPE with an elastomer, and a plastomer with polycarbonate. Particularly preferred polymeric containers comprise blends of polyethylene (PE) and PP with a low percentage (≦8 wt %) of PE.

The shape, style, and/or size of containers for use with the present invention is not important. For example, the container can be a bottle, a vial, or a syringe. According to a preferred embodiment, the container is preferably a “small volume” bottle. As used therein, the term “small volume” bottle shall mean a bottle of a size sufficient to hold a quantity of liquid medicine sufficient for 1-3 topical doses per day over 1-2 months, generally about 20 ml or less. By way of example, small volume containers include 2.5 ml, 5 ml, 10 ml and 15 ml sized bottles, adapted for topically administering eye drops. Small volume bottles made of LDPE are easier to squeeze than larger bottles, and oval bottles are easier to squeeze than round bottles. Accordingly, liquid preparations adapted for topical ophthalmic administration are preferably packaged in oval LDPE bottles.

Sorptive losses of prostaglandins, or derivatives or analogues thereof, may relatively be higher in containers with small volumes. The acceptable degree of sorption depends on e.g. the active ingredient, the type of material, and recommended storage conditions, such as temperature and storage time, for the pharmaceutical composition in question.

The container according to the invention has a surfactant barrier layer on an inner surface of the container wall. This layer comprises one or more suitable surfactants, and optionally other constituents. The surfactant barrier layer is applied by treating the plastic material that constitutes, or will constitute, the container with at least one surfactant. Thus, in the manufacturing of a suitable container, the plastic material is treated with the surfactant. Subsequently, the material is shaped into a desired container form, and the container is filled with a composition comprising prostaglandins. Optionally, the plastic material is first formed into a desired container form, and the plastic material of the container is treated with the surfactant. Finally, the container is filled with a composition comprising prostaglandins. By the term “treating” is meant either impregnation or coating or any other method, e.g. chemical bonding, known to add surfactants to a polymeric surface.

When employing the impregnation technique, the polymeric containers are impregnated with a solution containing one or more of the surfactants according to the invention in a solvent. The solvent may e.g. be aqueous-based, organic-based, or a combination of the two. The container may be impregnated by any method well known to those skilled in the art. A typical procedure comprises simply immersing the containers in the surfactant solution for an amount of time sufficient to produce the desired improvement and may be followed by a treating or drying or other curing step. Typical contact times generally range from about 10 seconds to about one hour or more, and will vary according to the concentration of the surfactant solution, the desired add-on, the thickness of the walls, the shape of the container etc. Suitable contact times may readily be determined given the teachings of his invention by those skilled in the art.

Examples of suitable solvents are mixtures comprising from about 55 to 65 vol % of methanol or acetone and from about 35 to 45 vol % water, or an isopropanol/water mixture in a preferred embodiment, the surfactant solution typically comprises from about 0.01% to about 25%, such as from about 1% to about 10%, (w/v) of one or more surfactants.

When employing the coating technique, the coating solution comprises a surfactant and a solvent. The solvent may, e.g., be aqueous-based, organic-based, or a combination of the two. In order to facilitate the drying step, the solvent should be one that can easily be evaporated. The concentration of the surfactant can vary quite widely depending upon the mode of application. The coating solution can be applied to the polymeric surface by conventional coating techniques, e.g. by spraying, dipping, brush-coating and the like. The drying of the coating solution can be performed at room temperature and ambient pressure. Higher or lower temperatures or pressures can also be used if desired. The drying process can also be performed with the aid of air flow. Whichever drying process is adopted, it needs to remove sufficient solvent so that the surfactant comes into direct contact with the polymeric surface so as to adhere to that surface. The polymeric surface can also be pretreated with suitable substances, e.g. ozone, oxygen, corona, plasma, etc, in order to improve the binding of the surfactant to the polymeric surface.

In general, the surfactant-containing mixtures may be applied by any suitable conventional means to the plastic material, e.g. spray application with fast-drying solvents, immersing, vapor deposition, etc.

The surfactants may be selected from the group comprising POE-80 sorbitan laurate, the Brij series, e.g. polyoxyethylene 20 stearyl ethers (also known as the Brij series of surfactants) (Brij 78), Brij 30, Brij 35, Brij 52, Brij 56, Brij 58, Brij 72, Brij 721, Brij 76, Brij 92, Brij 96, and Brij 98, the Tween®/Polysorbate series (poloxyethylene sorbitan esters), e.g. Tween® 20, Tween® 40, Tween® 60, and Tween® 80, phospholipids and their derivatives, ethoxylated alkyl phenol, pegylated hydrogenated castor oils (e.g. CREMOPHOR® EL, C CREMOPHOR® RH and CREMOPHOR® CO-40), stearoyl macrogol esters, glyceryl stearates, Estasan series (medium chain tri-glycerides), sorbitan fatty acid esters.

Packaging of a pharmaceutical composition comprising a prostaglandin, or an analogue or a derivative thereof, with the method according to the invention provides low sorptive losses of the prostaglandin, or the analogue or derivative thereof, to the inner surfaces of the container wall.

There is also provided a method of stabilizing a prostaglandin, or an analogue or a derivative thereof, contained in a pharmaceutical composition. The method comprises packaging the pharmaceutical composition in a plastic container having a surfactant barrier layer on an inner surface of the container wall. The surfactant barrier layer lining the container wall provides increased stability of the prostaglandin, or the analogue or derivative thereof, contained in the pharmaceutical composition. This method and the other methods according to the invention provide storage-stable compositions, suitable for long-term storage at room temperature.

In yet another aspect of the invention, there is provided a method of preventing sorptive loss of a prostaglandin, or an analogue or a derivative thereof, from a pharmaceutical composition to the wall of a plastic container containing the pharmaceutical composition. The method comprises packaging the pharmaceutical composition in the plastic container, said container having a surfactant barrier layer on an inner surface of the container wall.

In the above-described methods, it is preferred that the pharmaceutical composition is an aqueous liquid. Optionally, the pharmaceutical composition is for ophthalmic use.

In an advantageous embodiment of the medicinal product and the methods according to the invention, the pharmaceutical composition further comprises a surfactant, in order to help solubilize or disperse the prostaglandin in the composition. The presence of surfactant on the container walls according to the invention prevents sorption of the surfactant from the composition to the container walls. Such sorption could potentially result in precipitation of the PG due to decreased solubility.

In one embodiment, the surfactants in the pharmaceutical composition and on the inner surface of the container wall, respectively, have the same type of surface charge, i.e. both surfactants are either predominantly negatively or positively charged. Thereby, the PG/surfactant micelles are repelled by the surfactant-coated container walls according to the invention. Conveniently, a single type of surfactant can be used both in the pharmaceutical composition and on the inner surface of the container wall, respectively. The presence of surfactants both in the composition and on the container walls allow for low sorption of both the PG's and the surfactants in the composition to the container surfaces. Thereby, undesirable PG loss by sorption to the container wall and by precipitation is avoided or diminished.

The concentration of the prostaglandin, or analogue or derivative thereof, in the pharmaceutical composition depends on the particular prostaglandin, mode of administration, medical condition to be treated, etc. By way of example, the concentration of the prostaglandin, or analogue or derivative thereof, in the pharmaceutical composition may be in the range of 0.001%-0.5% (w/v), preferably 0.005%-0.1% (w/v). Useful prostaglandins, or analogues or derivatives thereof, in the method according to the invention are described above. Preferred prostaglandins, or analogue or derivative thereof, include 17-phenyl-18,19,20-trinor derivatives, 13,14-dihydro-17-phenyl-18,19,20-trinor derivatives, 16-phenoxy-17,18,19,20-tetranor derivatives, or 16-phenyl-17,18,19,20-tetranor derivatives.

In the above-described methods, it is preferred that the container is made of either LDPE, HDPE, and PP, or blends containing two or more of the following: LDPE, HDPE, PP, plastomers, and polycarbonates. A preferred polymeric material is a blend of: (a) 0.1-8 wt % of LDPE or HDPE, or a combination of LDPE and HDPE, and (b) 92-99.9 wt % of PP.

In the above-described methods, the surfactant barrier layer comprises at least one surfactant. Useful surfactants are described above. Preferred surfactants include Polysorbates (e.g. polysorbate 20 or 80), Brij series (e.g. Brij 35, 52 or 78) and hydrogenated pegylated castor oil derivatives e.g. CREMOPHOR® EL, CREMOPHOR® RH and CREMOPHOR® CO-40.

In a final aspect of the present invention, there is provided a novel use of a surfactant for preventing sorptive loss of an ingredient in a pharmaceutical composition comprising a prostaglandin, or an analogue or a derivative thereof, to a wall of a plastic container containing the pharmaceutical composition, by providing a surfactant barrier layer comprising said surfactant on an inner surface of the container wall.

The present invention will now be further illustrated by way of examples. The scope of this invention is not in any way intended to be limited to the specific embodiments disclosed in the following examples.

### EXAMPLES

### Example 1

The insides of one polyethylene container and one polypropylene container, each suitable for use as a container for prostaglandin compositions, are sprayed with a warm alcoholic solution of balsam and one or more surfactants, and cured at a temperature above room temperature (preferably 60° C. to 70° C.) for 10 minutes or longer. Suitable surfactants include Polysorbate 20 and Polysorbate 80; Brij 35, Brij 52, and Brij 78; and hydrogenated pegylated castor oil derivatives, e.g. CREMOPHOR® EL, CREMOPHOR® RH and CREMOPHOR® CO-40.

### Example 2

One polyethylene container and one polypropylene container, each suitable for use as a container for prostaglandin compositions, are completely immersed in a concentrated alcoholic solution of one or more surfactants for 1 hour or more and then air-dried at room temperature or higher. Suitable surfactants include Polysorbate 20 and Polysorbate 80; Brij 35, Brij 52, and Brij 78; and hydrogenated pegylated castor oil derivatives, e.g. CREMOPHOR® EL, CREMOPHOR® RH and CREMOPHOR® CO-40.

### Example 3

A concentrated solution of POE(23) lauryl ether and CREMOPHOR® CO-40 in alcohol is prepared, and polyethylene bottles are soaked in the solution overnight at room temperature. Subsequently, the bottles are inverted and drained of excess solution and then dried at room temperature in a laminar flow hood for 1 day.

A composition of 0.005% latanoprost in phosphate buffer at pH 6.0, containing 0.05% CREMOPHOR® CO-40 and 0.02% benzalkonium chloride, is prepared, and 2 ml of the composition is placed in the surfactant-treated and untreated (control), respectively, polyethylene containers. The filled containers are stored at 56° C. and their contents are sampled for latanoprost following 1, 2, 4, and 8 weeks of incubation. The concentrations of latanoprost remaining in the containers are expressed as a percentage of latanoprost in bulk solution, stored at 56° C. in glass vials.

### Example 4

Polyethylene bottles are pre-coated with polysorbate 80 (PS 80) as outlined in Example 1. The surfactant-coated polyethylene bottles are filled with:
(a) Unoprostone isopropyl 0.15% in phosphate buffer (pH 6) with 0.1% PS 80; or
(b) Latanoprost 0.005% in phosphate buffer (pH 6) with 0.05% CREMOPHOR® CO-40; or
(c) Fluprostenol isopropyl 0.005% in Tris buffer (pH 7) with 0.05% Brij 78,
   in order to stabilize the prostaglandins compositions.

### Example 5

Polyethylene and polypropylene bottles are pre-coated with PS 80 as outlined in Example 1. The surfactant-coated polyethylene or polypropylene bottles are filled with:
(a) Latanoprost 0.005% in phosphate buffer (pH 6) with 0.05% CREMOPHOR® CO-40; or
(b) Latanoprost 0.075% in phosphate buffer (pH 6) with 0.05% CREMOPHOR® CO-40; or
(c) Latanoprost 0.125% in phosphate buffer (pH 6) with 0.075% CREMOPHOR® CO-40,
   in order to stabilize the prostaglandin compositions.

### Example 6

Ophthalmic compositions comprising approximately 50 μg/ml latanoprost, and approximately 50 μg/ml latanoprost and 5 mg/ml timolol, respectively, are packaged in bottles. The bottles are made of clear low-density polyethylene with a clear low-density polyethylene dropper tip, a high-density polyethylene screw cap, and a clear low-density polyethylene overcap. The inner surface of the bottle walls are pretreated with one or more of the following surfactants: Polysorbate 20 and Polysorbate 80; Brij 35, Brij 52, and Brij 78; and hydrogenated pegylated castor oil derivatives, e.g. CREMOPHOR® EL, CREMOPHOR® RH and CREMOPHOR® CO-40. Upon storage of the bottles at room temperature, the concentrations of latanoprost and latanoprost/timolol, respectively, are favorably maintained in the products.

The examples and preparations provided above further illustrate and exemplify the compounds of the present invention and methods of preparing such compounds. It is to be understood that the scope of the present invention is not limited in any way by the scope of the preceeding examples and preparations.

1. A packaged medicinal product comprising:
a) a pharmaceutical composition comprising a prostaglandin, or an analogue or a derivative thereof; and
b) a plastic container having a surfactant barrier layer on an inner surface of the container wall, wherein the container is filled with the pharmaceutical composition.

2. A packaged medicinal product according to claim 1, wherein said pharmaceutical composition is an aqueous liquid.

3. A packaged medicinal product according to claim 1, wherein the concentration of said prostaglandin, or analogue or derivative thereof, in said pharmaceutical composition is in the range of 0.001%-0.5% (w/v).

4. A packaged medicinal product according to claim 3, wherein the concentration of said prostaglandin, or analogue or derivative thereof, in said pharmaceutical composition is in the range of 0.005%-0.1% (w/v).

5. A packaged medicinal product according to claim 1, wherein said prostaglandin, or analogue or derivative thereof, is selected from the group comprising physiologically acceptable derivatives of prostaglandin F (PGF), in which the omega chain has the formula (I): 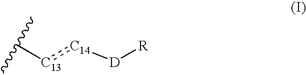
wherein
the bond between C₁₃ and C₁₄ is optionally a single bond or double bond;
D is a chain with 2-3 carbon atoms, optionally comprising at least one heteroatom selected from the group consisting of O, S, and N, and optionally substituted by one or more substituents selected from the group consisting of H, C₁-C₅ alkyl, halo, keto and hydroxy;
R is a
(i) phenyl group optionally substituted by one or more substituents selected from the group consisting of C₁-C₅ alkyl, C₁-C₄ alkoxy, trifluoromethyl, C₁-C₃ aliphatic acylamino, nitro, halo, and phenyl; or
(ii) an aromatic heterocyclic group having 5-6 ring atoms, selected from the group consisting of thiozol, imidazole, pyrrolidine, thiophene and oxazole, optionally substituted by one or more substituents selected from the group consisting of C₁-C₅ alkyl, C₁-C₄ alkoxy, trifluoromethyl, C₁-C₃ aliphatic acylamino, nitro, halo, and phenyl; or
(iii) C₃-C₇ cycloalkyl or C₃-C₇ cycloalkylene optionally substituted by C₁-C₅ alkyl.

6. A packaged medicinal product according to claim 5, wherein said prostaglandin, or analogue or derivative thereof, is selected from the group consisting of 17-phenyl-18,19,20-trinor derivatives, 13,14-dihydro-17-phenyl-18,19,20-tri nor derivatives, 16-phenoxy-17,18,19,20-tetranor derivatives, and 16-phenyl-17,18,19,20-tetranor derivatives.

7. A packaged medicinal product according to claim 1, wherein said plastic container is made of a polymeric material selected from the group consisting of low-density polyethylene (LDPE), high-density polyethylene (HDPE), polypropylene (PP) and blends thereof.

8. A packaged medicinal product according to claim 7, wherein said polymeric material is a blend of
(a) 0.1-8 wt % of LDPE or HDPE, or a combination of LDPE and HDPE, and
(b) 92-99.9 wt % of PP.

9. A packaged medicinal product according to claim 1, wherein said plastic container is a small volume bottle.

10. A packaged medicinal product according to claim 1, wherein said surfactant barrier layer comprises a surfactant selected from the group consisting of polyoxyethylene [20] sorbitan monolaurate or polyethylene sorbitan monooleate, sorbitan laurate, polyoxyethylene 20 stearyl ethers, poloxyethylene sorbitan esters, phospholipids and their derivatives, ethoxylated alkyl phenol, pegylated hydrogenated castor oils, stearoyl macrogol esters, glyceryl stearates, medium chain tri-glycerides, and sorbitan fatty acid esters.

11. A packaged medicinal product according to claim 10, wherein said surfactant is selected from the group consisting of polyoxyethylene [20] sorbitan monolaurate or polyethylene sorbitan monooleate, polyoxyethylene 20 stearyl ethers, poloxyethylene sorbitan esters, phospholipids and their derivatives, ethoxylated alkyl phenol, pegylated hydrogenated castor oils, stearoyl macrogol esters, glyceryl stearates, medium chain tri-glycerides, and sorbitan fatty acid esters.

12. A packaged medicinal product according to claims 1, wherein said pharmaceutical composition further comprises a surfactant.

13. A packaged medicinal product according to claim 12, wherein said surfactants in the pharmaceutical composition and on said inner surface of the container wall, respectively, have the same type of surface charge.

14. A packaged medicinal product according to claim 13, wherein a single type of surfactant is used both in the pharmaceutical composition and on said inner surface of the container wall, respectively.

15. A packaged medicinal product according to claim 1, which is for ophthalmic use.

16. A method of packaging a pharmaceutical composition comprising a prostaglandin, or an analogue or a derivative thereof, said method comprising the steps of:
(a) providing a plastic container having a surfactant barrier layer on an inner surface of the container wall; and
(b) filling the container with the pharmaceutical composition.

17. A method of stabilizing a prostaglandin, or an analogue or a derivative thereof, contained in a pharmaceutical composition, said method comprising packaging the pharmaceutical composition in a plastic container having a surfactant barrier layer on an inner surface of the container wall.

18. A method of preventing sorptive loss of a prostaglandin, or an analogue or a derivative thereof, from a pharmaceutical composition to the wall of a plastic container containing the pharmaceutical composition, said method comprising packaging the pharmaceutical composition in the plastic container, said container having a surfactant barrier layer on an inner surface of the container wall.

19. A method according to claim 16, wherein said pharmaceutical composition is an aqueous liquid.

20. A method according to claim 16, wherein the concentration of said prostaglandin, or analogue or derivative thereof, in said pharmaceutical composition is in the range of 0.001%-0.5% (w/v).

21. A method according to claim 20, wherein the concentration of said prostaglandin, or analogue or derivative thereof, in said pharmaceutical composition is in the range of 0.005%-0.1% (w/v).

22. A method according to claim 16, wherein said prostaglandin, or analogue or derivative thereof, is selected from the group comprising physiologically acceptable derivatives of PGF, in which the omega chain has the formula: 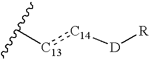
wherein
the bond between C₁₃ and C₁₄ is optionally a single bond or double bond;
D is a chain with 2-3 carbon atoms, optionally comprising at least one heteroatom selected from the group consisting of O, S, and N, and optionally substituted by one or more substituents selected from the group consisting of H, C₁-C₅ alkyl, halo, keto and hydroxy;
R is a
(i) phenyl group optionally substituted by one or more substituents selected from the group consisting of C₁-C₅ alkyl, C₁-C₄ alkoxy, trifluoromethyl, C₁-C₃ aliphatic acylamino, nitro, halo, and phenyl; or
(ii) an aromatic heterocyclic group having 5-6 ring atoms, selected from the group consisting of thiozol, imidazole, pyrrolidine, thiophene and oxazole, optionally substituted by one or more substituents selected from the group consisting of C₁-C₅ alkyl, C₁-C₄ alkoxy, trifluoromethyl, C₁-C₃ aliphatic acylamino, nitro, halo, and phenyl; or
(iii) C₃-C₇ cycloalkyl or C₃-C₇ cycloalkylene optionally substituted by C₁-C₅ alkyl.

23. A method according to claim 22, wherein said prostaglandin, or analogue or derivative thereof, is selected from the group consisting of 17-phenyl-18,19,20-trinor derivatives, 13,14-dihydro-17-phenyl-18,19,20-trinor derivatives, 16-phenoxy-17,18,19,20-trinor derivatives, and 16-phenyl-17,18,19,20-tetranor derivatives.

24. A method according to claim 16, wherein said container is made of a polymeric material selected from the group consisting of low-density polyethylene LDPE), high-density polyethylene (HDPE), polypropylene (PP) and blends thereof.

25. A method according to claim 24, wherein said polymeric material is a blend of:
a) 0.1-8 wt % of LDPE or HDPE, or a combination of LDPE and HDPE, and
b) 92-99.9 wt % of PP.

26. A method according to claim 16, wherein said container is a mall volume bottle.

27. A method according to claim 16, wherein said surfactant barrier layer comprises a surfactant selected from the group consisting of sorbitan laurate, polyoxyethylene 20 stearyl ethers, poloxyethylene sorbitan esters, phospholipids and their derivatives, ethoxylated alkyl phenol, pegylated hydrogenated castor oils, stearoyl macrogol esters, glyceryl stearates, medium chain tri-glycerides, and sorbitan fatty acid esters.

28. A method according to claim 27, wherein said surfactant is selected from the group consisting of polysorbates, Brij series, and hydrogenated pegylated castor oil derivatives.

29. A method according to any one of claims 16-28, wherein said pharmaceutical composition further comprises a surfactant.

30. A method according to claim 29, wherein said surfactants in the pharmaceutical composition and on said inner surface of the container wall, have the same type of surface charge.

31. A method according to claim 30, wherein a single type of surfactant is used both in the pharmaceutical composition and on said inner surface of the container wall.

32. A method according to claim 16, wherein said pharmaceutical composition is for ophthalmic use.

33. A method of using of a surfactant for preventing sorptive loss of an ingredient in a pharmaceutical composition comprising a prostaglandin, or an analogue or a derivative thereof comprising steps of:
a) packaging a pharmaceutical composition in the plastic container
b) having a wall of said plastic container contain the pharmaceutical composition,
c) providing a surfactant barrier layer comprising said surfactant on an inner surface of the container wall.
